(19) United States
(12) Patent Application Publication
(10) Pub. No.: US 2009/0056721 A1
(43) Pub. Date: Mar. 5, 2009

(54) Respiratory apparatus with mouthpiece

(76) Inventors: Andreanne Leboeuf, Montreal (CA); Louis-Philippe Fortier, Anjou (CA)

Correspondence Address: Ralph A. Dowell of DOWELL & DOWELL P.C., 2111 Eisenhower Ave, Suite 406, Alexandria, VA, 22314, US

(21) Appl. No.: 12/081,489

(22) Filed: Apr. 16, 2008

Related U.S. Application Data
(60) Provisional application No. 60/907,759, filed on Apr. 16, 2007.

(51) Int. Cl. A61M 16/04; A61M 16/00

(52) U.S. Cl. 128/207.14

(57) ABSTRACT

Disclosed herein is a respiratory apparatus that acts as a system for pre-oxygenation of a patient and for the opening of respiratory pathways of a patient. The apparatus includes a mouthpiece having a shield that is inserted in the mouth of the patient. The mouthpiece also includes an outer tubular member for receiving a universal connector which can be connected to an anesthetic device. The shield has an inner side opening which leads to the inside of the tubular member. This opening is configured to receive a connector mounted to a tracheal tube. The tracheal tube is placed within the patient. The universal connector and the tracheal connector are put into mutual fluid communication within the tubular member thereby providing gaseous fluid to flow from the universal connector to the tracheal tube which is in fluid communication with the patient's trachea. The shield also includes teeth abutments or bite members which are to be abutted by the teeth of the patient in order to keep their mouth open.

### CROSS-REFERENCE TO RELATED APPLICATIONS

This application requests priority on U.S. Provisional Application Ser. No. 60/907,759 filed on Apr. 16, 2007 and incorporated herein by reference in its entirety.

### FIELD OF THE INVENTION

The present invention relates to a respiratory apparatus. More specifically but not exclusively, the present invention is concerned with an apparatus, for pre-oxygenation and for opening of respiratory pathways, having an intra-oral mouthpiece.

### BACKGROUND OF THE INVENTION

Pre-oxygenation is an important part of the anaesthesia process. General anaesthesia is basically an association of medications that have three specific and complementary effects on a patient. Hypnotics, which cause loss of consciousness, analgesics, which relieve pain and curares which act as muscle relaxants and therefore prohibiting involuntary movement. However, curares and certain hypnotics can cause a loss in muscle tone resulting in a drooping of the palate which can obstruct the pharynx. When a more serious form of anesthesia is required, the curares involved may cause a complete loss in muscle tone in the patient (including all respiratory muscles).

FIG. 1 shows a patient P under normal conditions, the nasal cavity K is in communication with the larynx L and trachea T since the tongue G does not obstruct the respiratory pathway. Under anesthesia, illustrated in FIG. 2, tongue G is in a relaxed state and falls back causing an obstruction O between in the respiratory pathway between the nasal cavity K and the larynx L.

It therefore becomes essential to intube the patient in order to ensure mechanical ventilation. Unfortunately, intubation can only be performed once the patient is unconscious due to the rejection instinct stimulated by touching the glottis. Therefore, there is a short period, between the end of natural respiration and the beginning of mechanical ventilation, during which the patient receives no air (i.e. apnea). In order to avoid arterial desaturation and hypoxemia (oxygen deficiency) at this point, it is necessary to pre-oxygenate the patient by replacing the nitrogen in his lungs with oxygen.

Air is usually composed of 21% oxygen and 78% nitrogen (the remaining 1% being a mix of various gases, including carbon dioxide). Consequently, during respiration, the lungs retain these gases in similar proportions. During pre-oxygenation, the first step of general anesthesia, the patient is made to breathe pure oxygen. The extra oxygen will ensure proper oxygenation during apnea. A facial mask is often used for both pre-oxygenation and the administering of anesthetic gases. This mask must be properly insulated to avoid oxygen and anesthetic gases from leaking out; this could be dangerous for the personnel involved (for fire hazard for example), as well as the patient, and ensure complete pre-oxygenation. An insufficient pre-oxygenation may cause complications during medical intervention and even go as far as being fatal for the patient.

Technically, pre-oxygenation is carried out by making the patient breathe pure oxygen through an insulated face mask for three to four minutes. However, facial morphology being such an important factor, it is impossible to fully insolate the mask for most patients. Claustrophobia and nervousness in the patient may also make it difficult for the anesthesiologist to maintain the mask in place for a sufficient amount of time. A mask also enhances claustrophobia and pre-intervention stress.

In light of the foregoing, it is easy to conceive that pre-oxygenation is generally viewed as an unpleasant procedure and it therefore becomes all the more important that the mask used is comfortable for the patient.

Essentially, there is a major gap in the pre-oxygenation procedure due mainly to the insufficiencies presented by the facial mask. Facial morphology being so different from one individual to the next, it is virtually impossible to create a universal model. Also, one must take into account the various factors that could interfere with the mask's efficiency, such as facial malformations, missing teeth and facial hair. As pre-oxygenation is a crucial step in the process of anesthesia; it is important to overcome these problems. Other problems with masks are that during anesthesia the facial muscles are relaxed and the patient's cheeks may fall inwards, potentially causing oxygen and anesthetic gases to escape.

Pre-oxygenation and general anesthesia normally take place in the operating block with access to a properly serviced and maintained anesthetic gas delivery machine. In some minor cases, an anesthesiologist may go through the anesthesia process outside of the operating block where he would not have access to an anesthetic gas delivery machine and would have to use the oxygen outlets available everywhere in the hospital. In those cases, it would better if the device used was equipped with an expiratory valve and an oxygen connector. The valve should further be equipped with a cap to avoid polluting the ambient air with anesthetic gases. However, adding such equipment would make the device heavier and limit its functionality.

In such a device, dead space should be optimally reduced to avoid the patient breathing in the expiratory gases left over in the dead space. The air or gases contained in the devices may be considered fluids as they obey the same mechanical laws. This is pertinent in trying to avoid problems such as gas turbulence in the tubes or hollow areas, thus minimizing the energy required to move the gas through said tubes. In order to determine these factors, we must apply Poiseuille's law and determine the Reynolds number associated with the situation. The Reynolds number is the ratio of inertial forces (vₛρ) to viscous forces (μ/L) and is used for determining whether a flow will be laminar or turbulent. This number is a function of the length and diameter of the tube. For instance, a Reynolds number greater than 10 000 signifies a turbulent flow. Using this number, we can determine the maximum length and diameter of tubing to be used in a hospital setting. While Poiseuille's law allows us to describe a fluid's flow through a tube of specific diameter.

### OBJECTS OF THE INVENTION

An object of the present invention is to provide a respiratory apparatus.

An object of the present invention is to provide an improved apparatus for pre-oxygenation and for opening of respiratory pathways.

### SUMMARY OF THE INVENTION

In accordance with an aspect of the present invention, there is provided a respiratory apparatus for a patient comprising: a mouthpiece for being inserted within the mouth of the patient and comprising teeth abutments configured to be engaged by the teeth of the patient; and an outer airway portion being mountable to the mouthpiece for providing a gaseous fluid to the patient, wherein when the apparatus is assembled and mounted to the patient a contiguous pathway is provided from the outer airway portion to the respiratory system of the patient, the teeth abutments being inserted between the teeth so as to keep the jaw of the patient as open as when the jaw muscles are at rest.

In accordance with another aspect of the present invention, there is provided a mouthpiece for a respiratory apparatus for a patient, the mouthpiece provided for being inserted within the mouth of the patient and comprising: an outer surface and an opposite inner surface; teeth abutments extending from the inner surface and configured to be engaged by the teeth of the patient; and a coupling member extending from the outer surface and configured to be coupled to the respiratory apparatus for providing a gaseous fluid to the patient, wherein when the mouthpiece is coupled to the respiratory apparatus and mounted to the patient a contiguous pathway is provided from the respiratory apparatus to the respiratory system of the patient, the teeth abutments being inserted between the teeth so as to keep the jaw of the patient as open as when the jaw muscles are at rest.

In accordance with an aspect of the present invention there is provided a respiratory apparatus for a patient, the apparatus comprising: a mouthpiece for being inserted within the mouth of a patient, an outer airway portion being mountable to the mouthpiece and configured to receive and provide a gaseous fluid; and an inner airway portion being mountable to the mouthpiece configured to be inserted within the mouth of the patient so as to communicate with the respiratory system of the patient; wherein the mouthpiece comprises a coupling portion for providing the outer airway portion to be in fluid communication with the inner airway portion.

In accordance with another aspect of the present invention there is provided a respiratory apparatus for a patient, the apparatus comprising: a mouthpiece for being inserted within the mouth of a patient, an outer airway portion being mountable to the mouthpiece and configured to receive and provide a gaseous fluid; the mouthpiece comprising an opening for providing the outer airway portion to be in fluid communication with the respiratory system of the patient.

In accordance with a further aspect of the present invention there is provided mouthpiece for a respiratory apparatus for a patient, the mouthpiece comprising: a shield for being inserted between the teeth and the lips of the patient; a coupling portion on the outer side of the shield for receiving an outer airway portion of the apparatus configured to receive and provide a gaseous fluid; an opening on the inner side of the shield for providing fluid communication between the outer airway portion and the respiratory system of the patient and/or for communicating with an inner airway portion of the apparatus for being put into communication with the respiratory system of the patient, the mouthpiece coupling portion providing for the inner airway portion to be in fluid communication with the outer airway portion.

In a non-restrictive embodiment, there is provided a respiratory apparatus that acts as a system for pre-oxygenation of a patient and for opening of the respiratory pathways of this patient. The apparatus includes a mouthpiece having a shield that is inserted in the mouth of the patient. The mouthpiece also includes an outer tubular member for receiving a universal connector which can be connected to an anesthetic device. The shield has an inner side opening which leads to the inside of the tubular member. This opening is configured to receive a connector mounted to a tracheal tube. The tracheal tube is placed within the patient's mouth. The universal connector and the tracheal connector are put into mutual fluid communication within the tubular member thereby providing gaseous fluid to flow from the universal connector to the tracheal tube which is in fluid communication with the patient's trachea. The shield also includes bite members which are to be abutted by the teeth of the patient in order to keep their mouth open.

Other objects, advantages and features of the present invention will become more apparent upon reading of the following non-restrictive description of illustrative embodiments thereof, given by way of example only with reference to the accompanying drawings.

### BRIEF DESCRIPTION OF THE DRAWINGS

In the appended drawings, where like reference numerals denote like elements throughout, and where:

FIG. 1 is a schematic cross-sectional view of a patient's head showing a part of the respiratory system under normal conditions;

FIG. 2 is a schematic cross-sectional view of a patient's head showing a part of the respiratory system under anesthesia;

FIG. 3 is a back perspective disassembled view of the respiratory apparatus of the invention in accordance with a non-restrictive embodiment thereof;

FIG. 4 is a back perspective assembled view of the respiratory apparatus of the invention in accordance with a non-restrictive embodiment thereof;

FIGS. 5a and 5b are back perspective views of the mouthpiece of the invention in accordance with a non-restrictive embodiment thereof;

FIG. 6 is a back perspective view of the tracheal tube and tracheal connector of the invention in accordance with a non-restrictive embodiment thereof;

FIG. 7 is a back perspective view of the tracheal connector of the invention in accordance with a non-restrictive embodiment thereof;

FIG. 8a is a front elevational view of the universal connector of the invention in accordance with a non-restrictive embodiment thereof;

FIG. 8b is front perspective view of the universal connector of FIG. 8a;

FIG. 9 is a front perspective view of the nose clip of the invention in accordance with a non-restrictive embodiment thereof;

FIG. 10 is a schematic cross-sectional view of patient's head having the apparatus of FIG. 1 mounted thereto;

FIG. 11 is a schematic cross-sectional view of patient's head having the apparatus of FIG. 1, but without the inner airway portion thereof, mounted thereto; and

FIG. 12 is front elevational view of a variety of components of the apparatus of FIG. 1 in accordance with a non-restrictive embodiment of the invention.

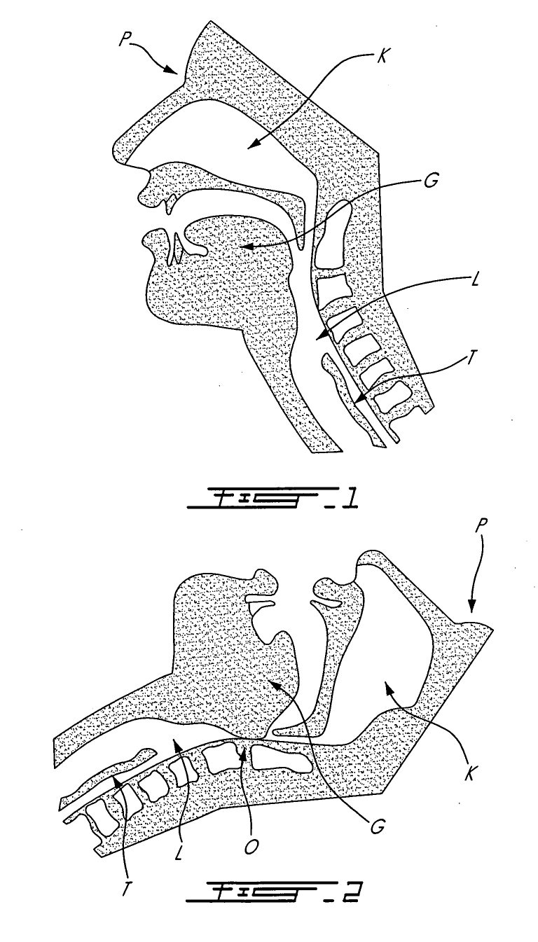

### DETAILED DESCRIPTION OF ILLUSTRATIVE EMBODIMENTS

If a device is to be placed in the patient's mouth, it must answer to certain constraints in order to avoid wounding the patient and maximize comfort. In one embodiment, the device must not extend for more than 5 mm in the area above and below the tooth, it must not extend beyond the first molar on the cheek side and the incisive on the tongue side and it must not interfere with the jaw muscles. The mouth must be maintained sufficiently opened for easy respiration and muscle comfort in the jaw, usually an opening of 1.5 to 2 cm between the teeth is adequate. If the patient does not have any teeth, it becomes necessary to compensate with a much thicker bite.

Generally stated the present invention provides a respiratory apparatus for a patient. The respiratory apparatus includes a mouthpiece which is inserted in the mouth of the patient. The mouthpiece includes teeth abutments configured to be engaged by the teeth of the patient. The apparatus includes an outer airway portion which is mounted to the mouthpiece. The outer airway portion includes a gaseous delivery device for providing a gaseous fluid to the patient. An inner airway portion is mounted to the mouthpiece and is in fluid communication with the respiratory system of the patient. When the apparatus is assembled and mounted to the patient a contiguous pathway is provided from the outer airway portion to the respiratory system of the patient. The teeth abutments are inserted between the patient's teeth so as to keep the jaw as open as when the jaw muscles are at rest.

With reference to the associated drawings non-restrictive illustrative embodiments of the present invention will now be described.

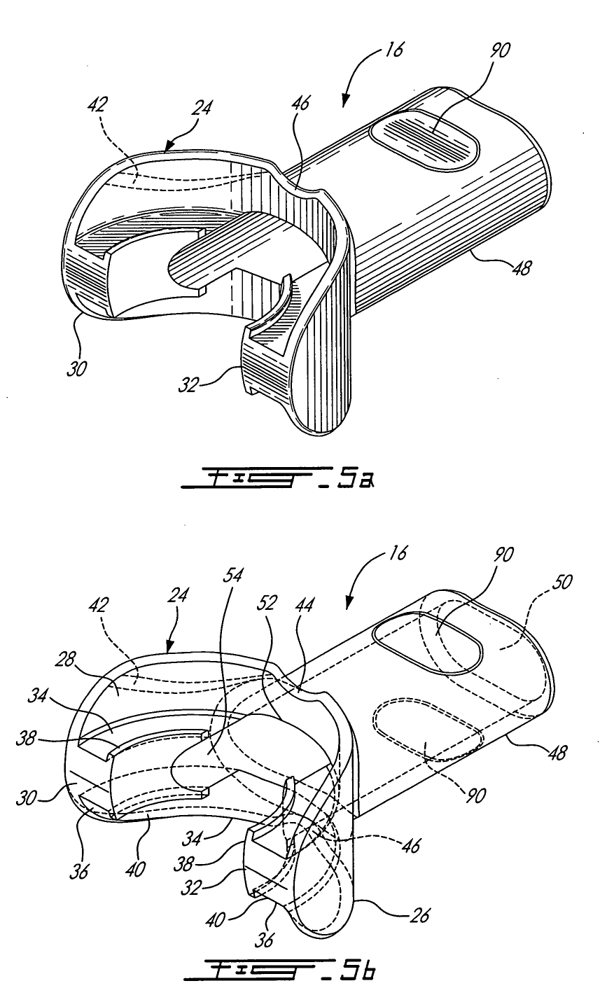

FIGS. 3 and 4 show a respiratory apparatus 10 in disassembled and assembled versions respectively in accordance with an illustrative embodiment of the present invention.

Generally, the apparatus 10 includes an outer airway portion 12 and an inner airway portion 14 separated by a mouthpiece 16 therebetween. More specifically, the apparatus 10 includes a mouthpiece 16 that is mountable to a tracheal tube 18 via a connector 20 thereby defining the inner portion 14 of the apparatus 10. The mouthpiece 16 is also mountable to a universal connector 22 for connecting the mouthpiece 16 to a gaseous fluid delivery device such as a respiratory, ventilation, or anaesthesia device and the like (not shown), thereby defining the outer portion 12 of the apparatus 10. Respiratory apparatus 10 can be used for pre-oxygenation for general anaesthesia.

The mouthpiece 16 is the centrepiece of apparatus 10 and is to be placed within the mouth of the patient for sealing thereof with the lips of the patient as will be described herein.

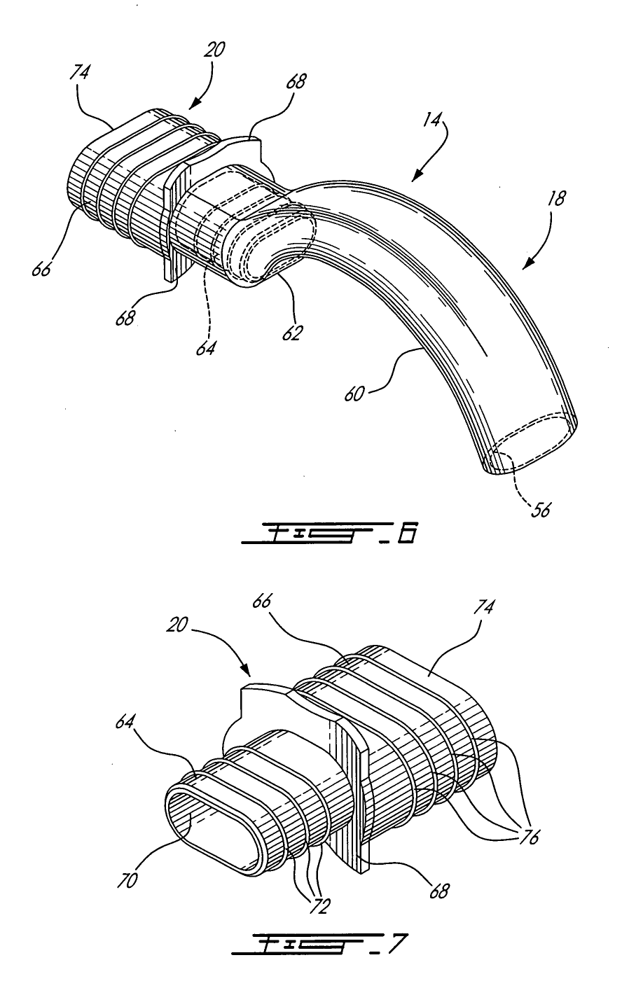

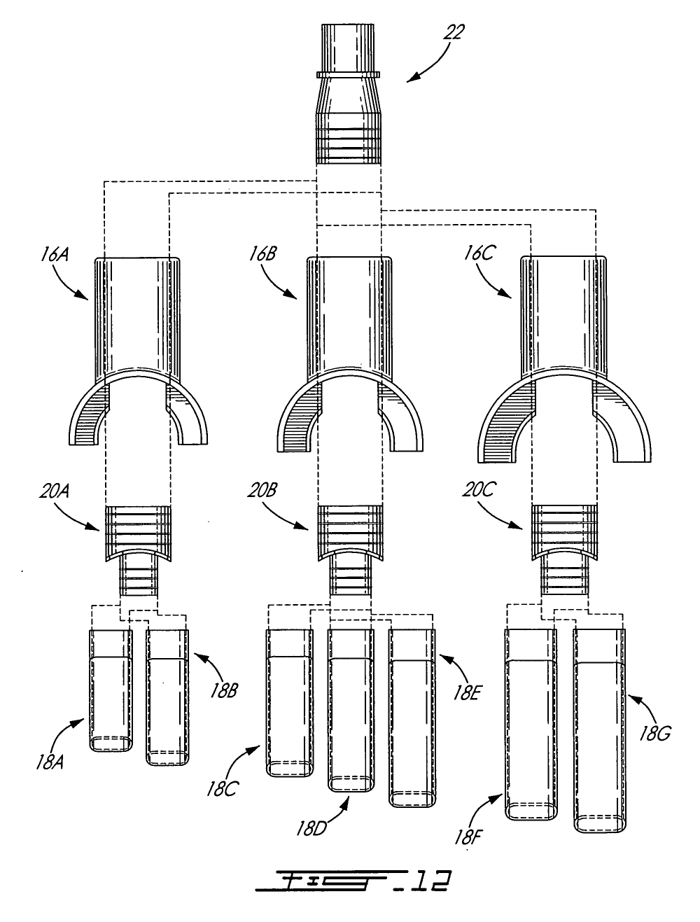

With reference to FIGS. 5a and 5b, the mouthpiece 16 comprises a generally U-shaped curved shield 24 having outer and inner surfaces 26 and 28, respectively. The inner surface 28 includes bite wings 30 and 32 defining respective top and bottom walls 34 and 36 which act as teeth abutments for the top and lower teeth D (see FIGS. 10 and 11) respectively. The thickness of each bite wing 30 and 32 provides for keeping the patient's jaws as open as when the jaw muscles are at rest. The inner surface 28 beyond and beneath the bite wings 30 and 32 is substantially perpendicular to top and bottom walls 34 and 36 and abuts the outer surface of the teeth along with the gums. The top and bottom walls 34 and 36 of each bite wing 34 and 36 also include short side edge walls 38 and 40, respectively, which may abut the inner surface of the teeth. The thickness of the bite wings 30 and 32 (defined by the distance between the inner surface 28 and a side edge wall 38 or 40) is such that they do not extend beyond the canines, thereby avoiding for the patient to feel that their mouth is full. The texture of the material (such as silicone for example) of the bite wings 30 and 32 provides for them to be bitten without scathing or scratching.

The shield 24 is flexible so as to be adjustable to the curvature of the patient's teeth and gums. The size of the shield 24 is designed to fully seal the patient's mouth between the teeth D and lips X1 and X2 (see FIGS. 10 and 11) while avoiding injury to the patient. Moreover, the inner surface 28 includes curved formations 42 so sized and configured as to fully merge with the outer surface of the teeth D thereby substantially sealing the patient's mouth. Hence, the inner surface 28 may comprise bumps separated by indentations.

The shield 24 includes small top and bottom notches 44 and 46 about the center of its U-shaped configuration. These notches 44 and 46 provide a space for the muscular filaments within the patient's mouth thereby avoiding injury.

The mouthpiece 16 also includes a coupling member such as outer short tubular member 48. The tubular member 48 includes a distal open end 50 and a proximal open end 52 which is positioned about the middle of the U-shaped shield 24 between the bite wings 30 and 32. The bite wings 30 and 32 include recessed walls 54 adjacent to this opening 52 for providing a clearance as will be described herein.

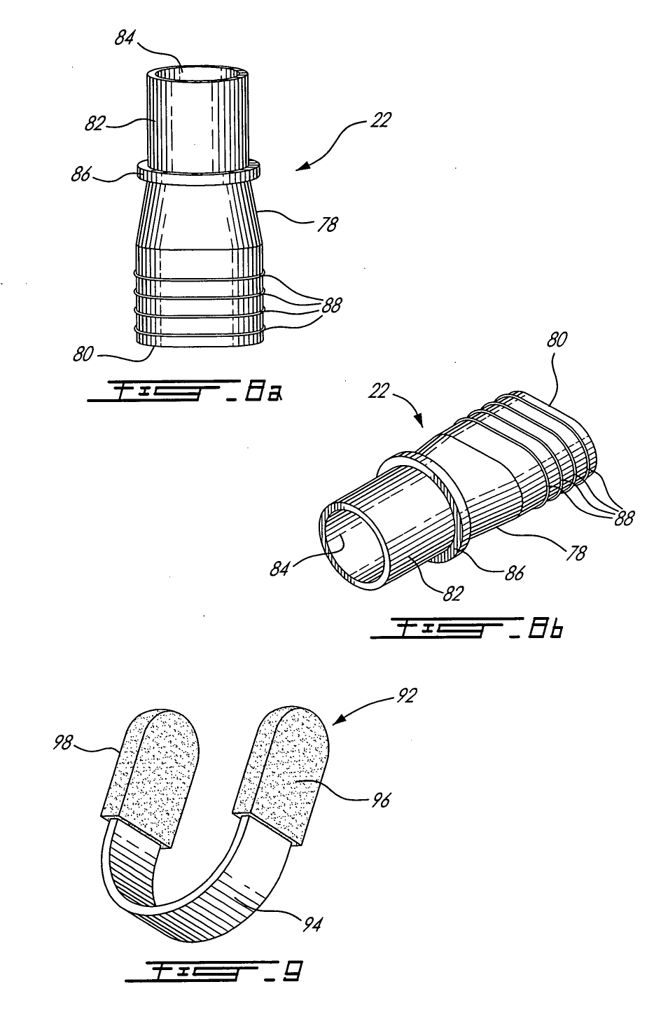

With respect to FIGS. 3, 4, 6, and 7 inner airway portion 14 including the tracheal tube 18 and connector 20 will be further described.

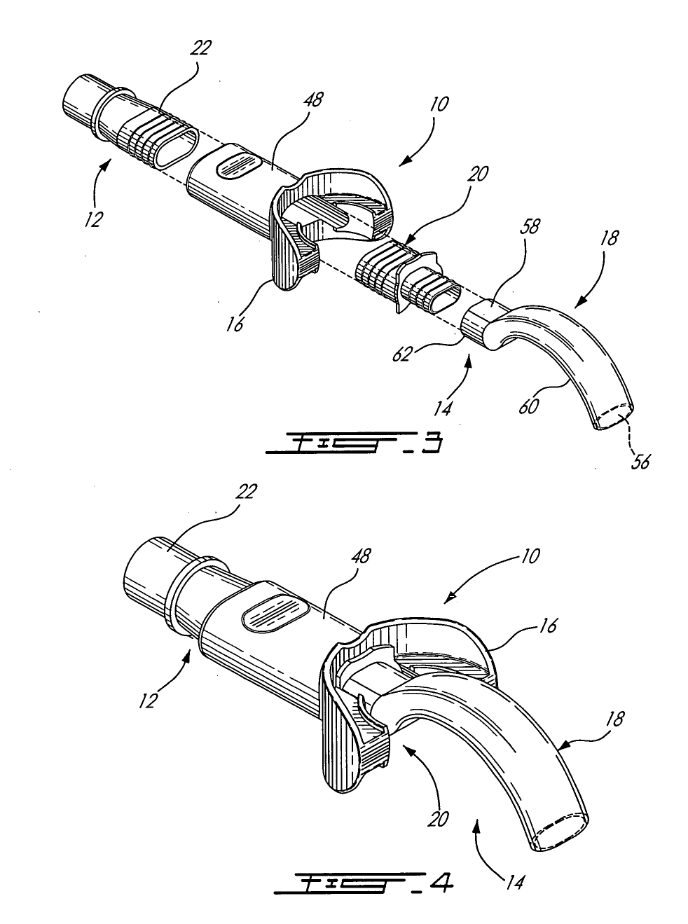

The tracheal tube 18, otherwise known as an endotracheal tube or a Guedel tube, is well known in the art yet, has been modified in accordance with the present invention. The tracheal tube 18 is used to maintain the respiratory pathways open when the respiratory muscles are in a relaxed state and to provide gaseous fluid to the respiratory system of the patient as will be explained herein. The tube 18 includes an open distal end 56 and an opposite open proximal end 58. The tube 18 includes a longer c-shaped curved portion 60 which is to be fitted deep into the mouth of the patient so that the distal end 56 is communication with the trachea T via the larynx L (see FIG. 10). The curvature of the tube 18 also keeps the back of the tongue G from falling back and blocking the respiratory pathway as was the case in FIG. 2. The tube 18 also includes a shorter connecting portion 62.

The connector 20 is a short tube including a smaller tracheal tube-connecting portion 64, an opposite and larger mouthpiece-connecting portion 66, and shoulder walls 68 therebetween. The tracheal tube-connecting portion 64 includes an open end 70 and is so configured and sized as to fit within the connecting portion 62 of tracheal tube 18 via opening 58. The mouthpiece-connecting portion 66 enters the portion 62 of tube 18 until the rim defined by opening 58 abuts the shoulder walls 68. The tracheal tube-connecting portion 64 includes external ridges 72 which provide for a friction fit within connecting portion 62 of tracheal tube 18. The shoulder walls 68 allow the user to grippingly disassemble the connector 20 from the tube 18. The mouthpiece-connecting portion 66 includes an open end 74 and is so sized and configured as to fit within tubular member 48 via opening 52. The mouthpiece-connecting portion 66 enters the tubular member 48 until the shoulder walls 68 abut the inner side 28 of shield 24 surrounding opening 52. As mentioned above, the recessed walls 52 of the bite wings 30 and 32 provide a clearance for the mouthpiece-connecting portion 66 and the shoulder walls 68 which have a curvature corresponding to the curvature of the shield 24 portion they abut. The mouthpiece-connecting portion 66 includes external ridges 76 which provide for a friction fit within tubular member 48. The connector 20 is so sized and configured as to provide the health care professional to easily manipulate it as well as adjust the tube 18.

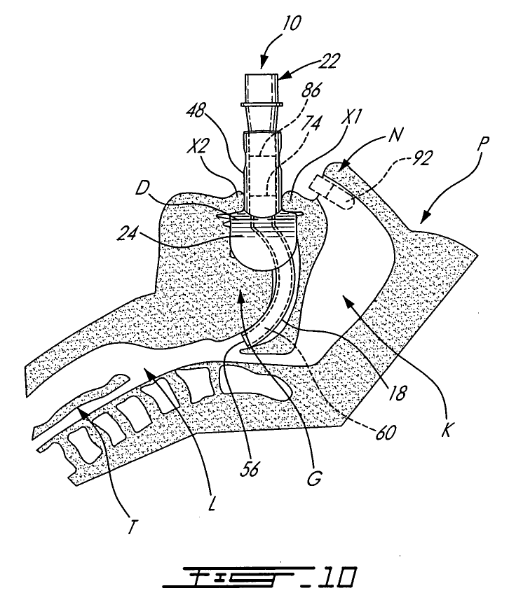

With reference to FIGS. 8a and 8b, the universal connector 22 is a short tubular member having a mouthpiece connecting portion 78, including an open end 80, and an opposite connecting portion 82 having a generally cylindrical configuration and including an open end 84. A ring shoulder 86 is positioned between portions 78 and 82. The mouthpiece connecting portion 78 is so sized and configured as to fit within tubular member 48 via opening 50. The mouthpiece connecting portion 78 enters the tubular member 48 until the ring shoulder 86 abuts the rim defined by opening 50. The mouthpiece-connecting portion 78 includes external ridges 88 which provide for a friction fit within tubular member 48. Ring shoulder 86 can also be used to grippingly remove the connector 22 from the mouthpiece tubular member 48.

When both connectors 20 and 22 are mounted to the mouthpiece 16, as described above, their respective openings 74 and 80 are in communication within tubular member 48 (see FIGS. 4 and 10). Connectors 20 and 22 add rigidity to the tubular member 48 when mounted thereto. The tubular member 48 also includes gripping portions 90 (see FIGS. 5a and 5b) allowing the user to more easily manipulate the mouthpiece 16 during assembling and disassembling operations.

The tubular connecting portion 82 of connector 22 is configured to be mounted to a variety of respiratory, ventilation, or anaesthesia devices and the like.

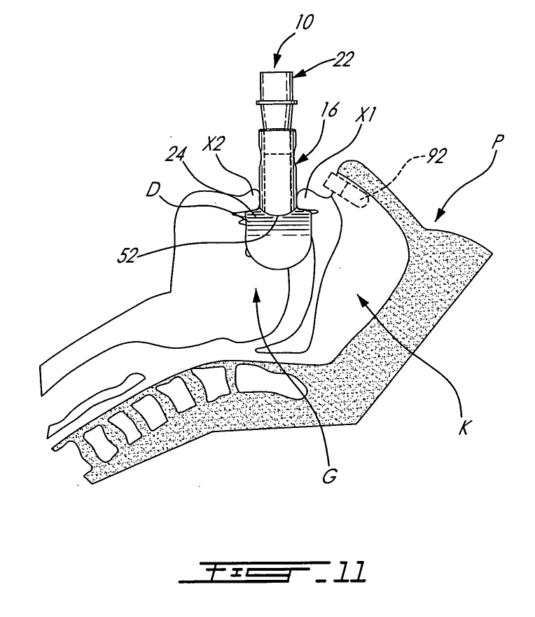

With respect to FIG. 9, a nose clip 92 is provided along with apparatus 10. The nose clip 92 includes a resilient and flexible body 94, made of iron in one example, with clipping ends 96 and 98. The clipping ends 96 and 98 can be coated with silicone for example. The nose clip is shown in operation, clipping the nose N of the patient P, in FIGS. 10 and 11, which closes the nasal cavity K.

Thereby, when the apparatus 10 is assembled as shown in FIG. 4 and mounted to a patient P as shown in FIG. 10, a contiguous pathway is provided between connector 22, mouthpiece tubular member 48 connector 20 and tracheal tube 18. As shown in FIG. 10, the lips X1 and X2 of the patient P rest on the outer surface 26 of shield 24. The teeth D abut the bite wings 30 and 32 providing the patient P with an opening in the mouth which allows a sufficient space for the tracheal tube 18 to be inserted into the mouth so as to communicate with the trachea T via the larynx L.

In another non-restrictive version of apparatus 10, the inner airway portion 14, including connector 20 and tracheal tube 18 have been removed and gaseous fluid is sent into the respiratory system of the patient P directly via opening 52 of the mouthpiece 16.

In many cases the positioning of the patient's head causing hyper-extension thereof and subluxation of the jaw are sufficient to permit passage of air. If there is obstruction of the patient's respiratory pathway, then the tracheal tube 18 can be used to open an obstruction (such as O in FIG. 2).

FIG. 12 shows a plurality of various component sizes and configurations of the apparatus 10. Hence, the mouthpiece 16 of the invention can at least be provided in small, medium, and large sizes such as 16A, 16B and 16C respectively; conveniently the tint of the mouthpiece 16 is darkened as the size increases. Similarly, the tracheal connector 20 can at least be provided in small, medium, and large sizes such as 20A, 20B and 20C; conveniently the size of the connector 20 is color coded. Various sizes of the tracheal tube 18 can be provided such as 18A, 18B, 18C, 18D, 18E, 18F, 18G which are identified in numerical order from the smallest size, such as 18A, to the largest size, such as 18G.

Of course the apparatus 10 and the components thereof can be modified in various ways as is understood by the skilled artisan. In the following list, are some non-exhaustive and non-compulsory elements that should be taken into consideration when designing an apparatus 10 in accordance with the present invention:
the patient's comfort;
avoiding too much material thus limiting nausea;
muscle ligation filaments;
the minimum and maximum areas to cover to ensure both comfort and functionality;
the complete or partial absence of teeth;
the possibility of crooked teeth;
the texture of the materials;
minimizing visual obstruction of the patient (thereby choosing shorter and smaller pieces visible to the patient for example);
efficiency (i.e. substantially avoiding mistakes on the physician's part;
allowing for the integration of related techniques;
versatility of the components;
interchangeable pieces;
manufacturing costs;
relative simplicity of components' shape;
limited technological need allowing for reasonable pricing, among other considerations.

The components of apparatus are relatively small in size. For example, connector 20 and tube 18 have a generally flattened tubular configuration that is small enough to comfortably fit into the patient's mouth and throat. Inner airway portions 14 of the invention will be so designed as to not cause any unwanted gagging of the patent and as such fit comfortably in the patient's mouth without taking too much unnecessary space. The intra-oral components of apparatus 10 are designed to meet dental and oral standards.

The connector 20, the tubular member 48, the connector 22 and the tube 18 are tightly fitted into one another as described above so as to avoid any part thereof from coming off during operation as well as to avoid any gaseous leaks from the apparatus 10. As mentioned above the shield 24 seals the patient's mouth between the teeth, gums and lips hence avoiding any gaseous leaks from the mouth as well.

An advantage of the present apparatus 10 is that it easy to handle for the health care professional, such as an anesthesiologist. The health care professional will only need one hand to lightly support the apparatus 10 on the patient while ventilating the patient with the other hand. The apparatus 10 facilitates the general anesthesia process since if there is a need for further oxygenation, the apparatus 10 can provide this further oxygenation with very little manipulation as compared to facial masks for example.

The components of apparatus 10 can be made of a variety of materials such as for example polymeric materials including silicones or polysiloxanes (such as polydimethylsiloxane or PDMS for example), polycarbonates and the like as well as combinations thereof to name only a few non-limiting examples. When choosing materials for manufacturing an apparatus 10, the skilled artisan should take the flexibility and/or malleability of the mouthpiece 16 into consideration which provides comfort to the patient. Other considerations include: using anti-allergenic materials, material transparency, sterilisable materials; material durability; ability of material to be colored; chemical stability of materials.

The various connectors of the invention may be made of polycarbonates. Polycarbonates are rigid, choc-resistant, sterilisable, transparent and medically approved materials.

The various features and characteristics of the various components of apparatus 10 can be combined in a variety of ways in order to provide for other designs and configurations within the scope of the present invention.

It is to be understood that the invention is not limited in its application to the details of construction and parts illustrated in the accompanying drawings and described hereinabove. The invention is capable of other embodiments and of being practised in various ways. It is also to be understood that the phraseology or terminology used herein is for the purpose of description and not limitation. Hence, although the present invention has been described hereinabove by way of embodiments thereof, it can be modified, without departing from the spirit, scope and nature of the subject invention.

What is claimed is:

1. A respiratory apparatus for a patient comprising:
a mouthpiece for being inserted within the mouth of the patient and comprising teeth abutments configured to be engaged by the teeth of the patient; and
an outer airway portion being mountable to said mouthpiece for providing a gaseous fluid to the patent,
wherein when said apparatus is assembled and mounted to the patient a contiguous pathway is provided from said outer airway portion to the respiratory system of the patient, said teeth abutments being inserted between the teeth so as to keep the jaw of the patient as open as when the jaw muscles are at rest.

2. A respiratory apparatus according to claim 1, wherein said teeth abutment comprise bite wings.

3. A respiratory apparatus according to claim 2, wherein said bite wings are spaced apart, said mouthpiece comprising and opening positioned between said spaced apart bite wings for being in fluid communication with said outer airway portion and the respiratory system of the patient.

4. A respiratory apparatus according to claim 3, wherein said bite wings comprise respective recessed walls being adjacent to said opening for providing a clearance for an inner airway portion.

5. A respiratory apparatus according to claim 2, wherein said bite wings define respective top and bottom walls for being engaged by the patient's upper and lower teeth respectively.

6. A respiratory apparatus according to claim 5, wherein said top and bottom walls comprise ridges for abutting the inner surfaces of the patient's teeth.

7. A respiratory apparatus according to claim 2, wherein the thickness of said bite wings is such that they do not extend beyond the patient's canines.

8. A respiratory apparatus according to claim 1, wherein said teeth abutments are made of a material that provides for substantially avoiding scathing or scratching when bitten.

9. A respiratory apparatus according to claim 1, wherein said mouthpiece further comprises a curved shield comprising inner and outer surfaces, said teeth abutments extending from said inner surface.

10. A respiratory apparatus according to claim 9, wherein said shield includes top and bottom notches for providing a space for the patient's muscular filaments when inserted in the patient's mouth.

11. A respiratory apparatus according to claim 9, wherein said shield is so flexible as to be adjustable to the curvature of the patient's teeth and gums.

12. A respiratory apparatus according to claim 9, wherein said shield is so configured and sized as to seal the patient's mouth between the teeth and the gums when inserted therein.

13. A respiratory apparatus according to claim 9, wherein said shield comprises inner curved formations so sized and configured as to merge with the outer surface of the patient's teeth when inserted in the patient's mouth.

14. A respiratory apparatus according to claim 1, wherein said mouthpiece further comprises a coupling member for being coupled to said outer airway portion.

15. A respiratory apparatus according to claim 14, wherein said coupling member comprises an outer tubular member having distal and proximal open ends.

16. A respiratory apparatus according to claim 15, wherein said outer tubular member comprises a gripping portion.

17. A respiratory apparatus according to claim 14, wherein said outer airway portion comprises a connector for being coupled to said coupling member, said connector being in fluid communication with a gaseous fluid delivery device.

18. A respiratory apparatus according to claim 17, wherein said connector is mountable within said coupling member via a distal opening thereof.

19. A respiratory apparatus according to claim 18, wherein said connector comprises a shoulder for abutting a rim defined by said coupling member opening.

20. A respiratory apparatus according to claim 1, further comprising an inner airway portion being mountable to said mouthpiece for being inserted within the mouth of the patient so as to be in fluid communication with the respiratory system of the patient and with said outer airway portion.

21. A respiratory apparatus according to claim 20, wherein said inner airway portion comprises a tracheal tube for maintaining the respiratory pathway of the patient open when the respiratory muscles are in a relaxed state and for providing gaseous fluid to the respiratory system of the patient.

22. A respiratory apparatus according to claim 21, wherein said tracheal tube comprises a curved portion having a distal end opening for being fitted deep within the mouth so that said distal end is in fluid communication with the patient's trachea.

23. A respiratory apparatus according to claim 22, wherein said curved portion provides for keeping the patient's tongue from blocking the respiratory pathway.

24. A respiratory apparatus according to claim 21, wherein said inner airway portion further comprises a connector for coupling said tracheal tube to said mouthpiece.

25. A respiratory apparatus according to claim 24, wherein said mouthpiece comprises a coupling member, said connector comprising a tracheal-tube connecting portion, a mouthpiece-connecting portion.

26. A respiratory apparatus according to claim 24, wherein said tracheal-tube connecting portion is fitted within said tracheal tube and said mouthpiece-connecting portion is fitted within said coupling member.

27. A mouthpiece for a respiratory apparatus for a patient, said mouthpiece provided for being inserted within the mouth of the patient and comprising:
an outer surface and an opposite inner surface;
teeth abutments extending from said inner surface and configured to be engaged by the teeth of the patient; and
a coupling member extending from said outer surface and configured to be coupled to the respiratory apparatus for providing a gaseous fluid to the patient,
wherein when said mouthpiece is coupled to the respiratory apparatus and mounted to the patient a contiguous pathway is provided from said respiratory apparatus to the respiratory system of the patient, said teeth abutments being inserted between the teeth so as to keep the jaw of the patient as open as when the jaw muscles are at rest.

28. A mouthpiece according to claim 27, wherein said teeth abutment comprise bite wings.

29. A mouthpiece according to claim 28, wherein said bite wings are spaced apart, said mouthpiece comprising and opening positioned between said spaced apart bite wings for being in fluid communication with the respiratory system of the patient.

30. A mouthpiece according to claim 29, wherein said bite wings comprise respective recessed walls being adjacent to said opening for providing a clearance for an inner airway portion.

31. A mouthpiece according to claim 28, wherein said bite wings define respective top and bottom walls for being engaged by the patient's upper and lower teeth respectively.

32. A mouthpiece according to claim 31, wherein said top and bottom walls comprise ridges for abutting the inner surfaces of the patient's teeth.

33. A mouthpiece according to claim 28, wherein the thickness of said bite wings is such that they do not extend beyond the patient's canines.

34. A mouthpiece according to claim 27, wherein said teeth abutments are made of a material that provides for substantially avoiding scathing or scratching when bitten.

35. A mouthpiece according to claim 27, wherein said mouthpiece further comprises a curved shield comprising inner and outer surfaces, said teeth abutments extending from said inner surface.

36. A mouthpiece according to claim 35, wherein said shield includes top and bottom notches for providing a space for the patient's muscular filaments when inserted in the patient's mouth.

37. A mouthpiece according to claim 35, wherein said shield is so flexible as to be adjustable to the curvature of the patient's teeth and gums.

38. A mouthpiece according to claim 35, wherein said shield is so configured and sized as to seal the patient's mouth between the teeth and the gums when inserted therein.

39. A mouthpiece according to claim 35, wherein said shield comprises inner curved formations so sized and configured as to merge with the outer surface of the patient's teeth when inserted in the patient's mouth.

40. A mouthpiece according to claim 27, wherein said coupling member comprises an outer tubular member having distal and proximal open ends.

41. A mouthpiece according to claim 40, wherein said outer tubular member comprises a gripping portion.
